(19) United States
(12) Patent Application Publication
(10) Pub. No.: US 2015/0265179 A1
(43) Pub. Date: Sep. 24, 2015

(54) Assisting Navigation of a Medical Insert Inside a Patient

(71) Applicant: Sham S. Lonkadi, Bangalore (IN)

(72) Inventor: Sham S. Lonkadi, Bangalore (IN)

(21) Appl. No.: 14/224,038

(22) Filed: Mar. 24, 2014

(51) Int. Cl. A61B 5/06; A61B 6/12

(52) U.S. Cl. CPC A61B 5/061; A61B 6/12

(57) ABSTRACT

A device and a method for assisting navigation of a medical insert inside a body of a patient are presented. An imaging unit generates image signals by sensing a field of view of the imaging unit. An image processing unit receives the image signals from the imaging unit and includes a position determining module to determine a position of the medical insert relative to a desired area within the field of view. A control module generates a control signal based on the position of the medical insert relative to the desired area. The position of the medical insert relative to the desired area is determined by using the image signals. A drive unit receives the control signal from the image processing unit and moves the field of view in accordance with the control signal such that the medical insert is maintained within the desired area.

### FIELD

The present embodiments relate to devices and methods for assisting navigation of medical inserts inside a body of a patient.

### BACKGROUND

In most modern invasive medical procedures (e.g., angiography, biopsy, etc.) that require insertion of a medical invasive device or a medical insert such as catheter, guide wire, biopsy needle, endoscope, pacemaker, etc., inside a patient's body, the movement of the medical insert inside the body of the patient is tracked. To facilitate proper navigation of the medical insert, the medical insert inside the body of the patient is continuously visualized on a display screen or exam room display monitor for viewing by a physician who is navigating the medical insert.

As the medical insert is advanced by the physician inside the body of the patient from one position to another, the imaging modality that is rendering images to the exam room monitor is focused such that the medical insert remains within a field of view of the imaging modality. Hence, during the navigation of the medical insert, the imaging modality (e.g., a C-Arm of an X-Ray machine) is moved continuously or intermittently to maintain the medical insert inside the field of view of the imaging modality.

The movement of the imaging modality is performed by an operator of the imaging modality, such as a technician who moves the C-Arm to maintain the medical insert in its field of view as and when the medical insert is moved or navigated inside the patient's body. For accurately moving the imaging modality, individual skills and expertise of the technician are instrumental. At all times during the navigational procedure, the technician is required to be alert, attentive and focused towards the exam room monitor. Thus, there is a chance of manual error in the present scenario.

Though usually the exam room monitor is arranged such that the physician and the technician both have a direct view of the exam room monitor, there exist scenarios, depending on the layout scheme of the exam room or depending on a special position the physician needs to assume to perform insertion and navigation of the medical insert, in which the technician may not have direct view of the exam room monitor. In such scenarios, the physician monitors the movement of the medical insert by looking at the exam room monitor and provides instructions (e.g., through verbal or visual cues) to the technician to move the imaging modality whenever the medical insert moves out or is about to move out of the field of view or is required to be centered in the field of view of the imaging modality. The technician, without the direct view of the exam room display screen, is unaware of the position of the medical insert and hence relies on the inputs provided by the physician to position the imaging modality, for example, by rotating the C-Arm. This further introduces chances of manual error because the communication between the physician and the technician is prone to human error and may lead to confusion and thus improper movement of the imaging modality. Chances of the medical insert moving out of the field of view of the imaging modality are increased when the navigation is being performed at a zoomed display on the exam room monitor.

Additionally, the navigation is to be performed as quickly as possible to provide that the patient is subjected to minimal exposure to radiations from the imaging modality.

### SUMMARY AND DESCRIPTION

It is desirable to develop a technique for assisting navigation of a medical insert inside a body of a patient in which chances of human error are reduced. The technique is to aid navigation of the medical insert inside the body of the patient without requiring human intervention to move the imaging modality such that the medical insert is maintained in the field of view of the imaging modality. This will provide error free navigation of the medical insert and faster navigation times and thus lesser radiation exposures of the patient.

The scope of the present invention is defined solely by the appended claims and is not affected to any degree by the statements within this summary.

The present embodiments may obviate one or more of the drawbacks or limitations in the related art. For example, an improved technique for assisting or aiding navigation of a medical insert inside a body of a patient is provided.

As another example, a technique for assisting or aiding navigation of a medical insert inside a body of a patient that requires minimal manual interventions and obviates high reliance on skill and experience of an operator or a technician is provided.

As yet another example, a technique for assisting or aiding navigation of a medical insert inside a body of a patient that facilitates the performance of the navigation procedure with reduction in radiation exposure of the patient is provided.

According to one embodiment, a medical device for assisting navigation of a medical insert inside a body of a patient is presented. The medical device includes an imaging unit, an image processing unit and a drive unit. The imaging unit, having a field of view, is configured to generate image signals by sensing the field of view. The image processing unit is in communication with the imaging unit and receives the image signals from the imaging unit. The image processing unit includes a position determining module and a control module. The position determining module is configured to determine a position of the medical insert relative to a desired area within the field of view of the imaging unit. The position of the medical insert relative to the desired area is determined by the position determining module by using the image signals. The control module is configured to generate a control signal based on the position of the medical insert relative to the desired area within the field of view as determined by the position determining module. The drive unit is in communication with the image processing unit. The drive unit receives the control signal from the image processing unit and is configured to move the field of view of the imaging unit in accordance with the control signal such that the medical insert is maintained within the desired area.

In one embodiment, the image signals may include a series of images of the medical insert inside the body of the patient at different positions within the field of view. In another embodiment, the position determining module is configured to determine a change in position of the medical insert relative to the desired area within the field of view of the imaging unit. The change in position of the medical insert relative to the desired area is determined by the position determining module by using at least a first image and a second image of the series. The control module is configured to generate the control signal based on the change in position of the medical insert relative to the desired area within the field of view as determined by the position determining module.

In another embodiment of the medical device, the position determining module is configured to detect a first position of the medical insert within the field of view from the first image of the series, to detect a second position of the medical insert within the field of view from the second image of the series, and to compare the first position and the second position to determine the change in position of the medical insert relative to the desired area within the field of view.

According to another embodiment, a method for assisting navigation of a medical insert inside a body of a patient is presented. In the method, image signals are generated by sensing a field of view of an imaging unit. Subsequently, a position of the medical insert relative to a desired area within the field of view is determined by using the image signals so generated. A control signal is generated based on the position of the medical insert relative to the desired area within the field of view. In the method, the field of view of the imaging unit is automatically moved in accordance with the control signal such that the medical insert is maintained within the desired area.

In one embodiment, the image signals may include a series of images of the medical insert inside the body of the patient at different positions within the field of view. In another embodiment of the method, determining the position of the medical insert relative to the desired area within the field of view includes determining a change in position of the medical insert relative to the desired area within the field of view by using at least a first image and a second image of the series. In the embodiment, the control signal is generated based on the change in position of the medical insert relative to the desired area within the field of view.

In another embodiment of the method, in determining the change in position of the medical insert, a first position of the medical insert within the field of view is detected from the first image of the series, and a second position of the medical insert within the field of view is detected from the second image of the series. Subsequently, the first position and the second position are compared to determine the change in position of the medical insert relative to the desired area within the field of view.

The present technique has several advantages. The present technique provides that the medical insert is continuously or intermittently maintained within the desired area in the field of view of the imaging unit and thus provides that the medical insert is available for visualization of a physician performing the navigation procedure. The present technique results in minimization of human intervention, and thus reducing manual errors and reliance on skill and expertise of a technician to aim the imaging unit such that the medical insert is maintained within the desired area in its field of view. The technique results in expeditious performance of the navigation procedure and thus provides that exposure of the patient to radiations from the imaging unit is decreased. The present technique also reduces the requirement of having technicians to control the movement of the imaging modality during the navigation procedure and thus makes the navigation procedure simple and economical.

### BRIEF DESCRIPTION OF THE DRAWINGS

FIG. 1 is a schematic representation of an exemplary embodiment of a medical device for assisting navigation of a medical insert inside a body of a patient;

FIG. 2 is a schematic representation of another exemplary embodiment of the medical device;

FIG. 3 is a schematic representation of an exemplary embodiment of an image depicting a field of view and a desired area within the field of view;

FIGS. 4, 5, 6, 7 and 8 schematically represent exemplary embodiments of images depicting the field of view and the medical insert within the body of the patient at different positions relative to the desired area;

FIG. 9 is a schematic representation of yet another exemplary embodiment of the medical device depicting a motion sensing unit;

FIG. 10 is a flow diagram depicting an exemplary embodiment of a method for assisting navigation of the medical insert inside the body of the patient; and

FIG. 11 is a flow diagram depicting another exemplary embodiment of the method.

Repeat use of reference characters in the present specification and drawings is intended to represent same or analogous features or elements of the technique.

### DETAILED DESCRIPTION

Reference now will be made in detail to various embodiments of the technique, one or more examples of which are set forth below. Each example is provided by way of explanation of the technique, not limitation of the technique. Various modifications and variations, as may be perceived by a person skilled in the art, may be made in the present technique without departing from the scope or spirit of the technique. Features illustrated or described as part of one embodiment, may be used on another embodiment. Thus, it is intended that the present technique covers such modifications and variations as come within the scope of the appended claims and their equivalents.

Although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or” includes any and all combinations of one or more of the associated listed items.

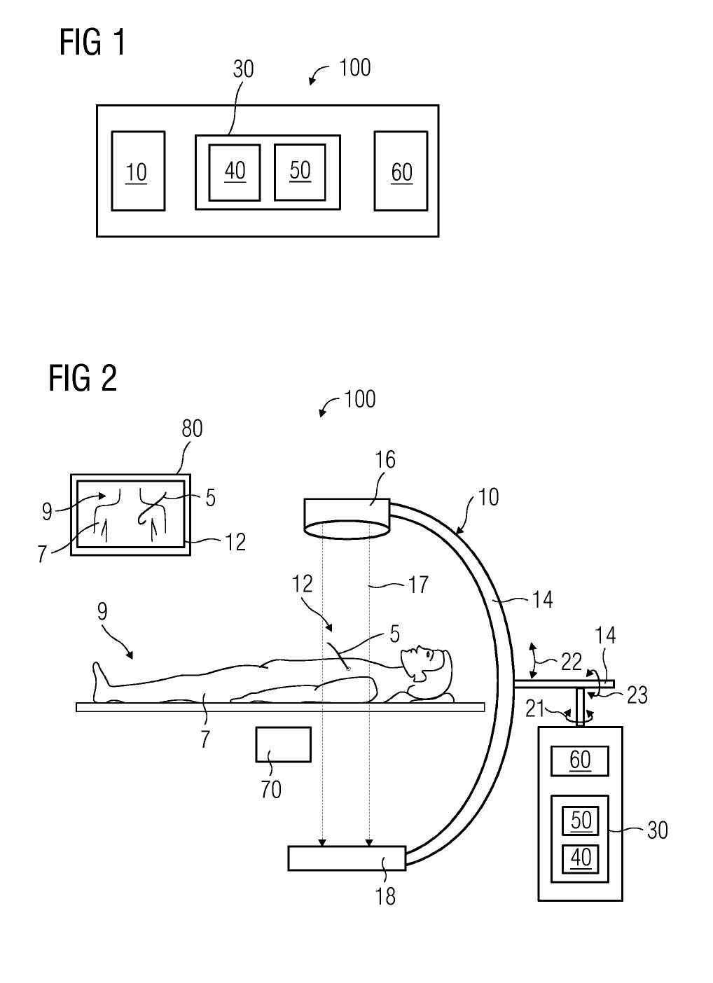

FIGS. 1 and 2 schematically represent an exemplary embodiment of a medical device 100 for assisting navigation of a medical insert 5 inside a body 7 of a patient 9, in accordance with the aspects of the present technique.

The ‘medical insert’ 5, as used herein, includes, but is not limited to, a catheter, a guide wire, a biopsy needle, an endoscope, a pacemaker, and other medical invasive probes used in medical procedures like angioplasty, pacemaker insertion, ablation therapy, catheterization, surgical cardiac electrophysiology, etc. The term ‘navigation’, as used herein, includes intravascular and non-vascular navigation of the medical insert 5. For the purposes of explanation and not for limitation, the medical insert 5 is shown as a guide wire in the accompanying FIGS.

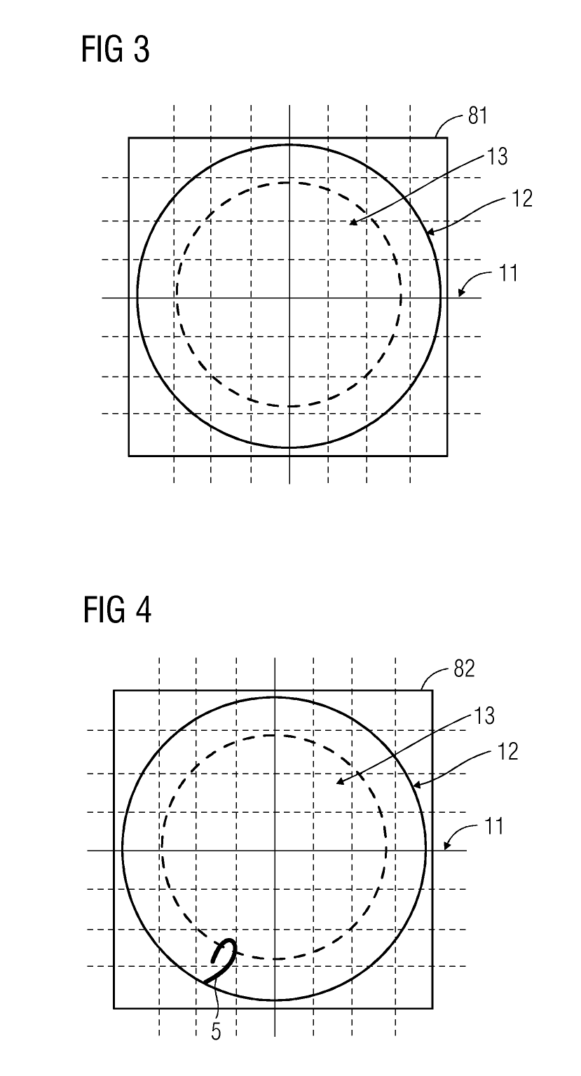

The medical device 100 includes an imaging unit 10 having a field of view 12, an image processing unit 30 and a drive unit 60. The imaging unit 10 is configured to generate image signals by sensing the field of view 12. The imaging unit 10 may be, but is not limited to, a fluoroscopic imaging device such as a C-Arm based X-Ray system. FIG. 3 is a schematic representation of an exemplary embodiment of an image 81 depicting the field of view 12 and a desired area 13 within the field of view 12. The image 81 is an exemplary image and does not show the medical insert 5. Though in FIG. 3 and the other accompanying FIGS, the field of view 12 and the desired area 13 are shown as circular, the field of view 12 and/or the desired area 13 may, however, be of any geometrical shape such as a rectangle or a square.

As shown in the exemplary embodiment depicted in FIG. 2, the imaging unit 10 may include a C-Arm mount 14. A radiation source 16, such as an X-ray emitter, is positioned at one end of the C-Arm mount 14. An image acquisition module 18, such as a radiographic receptor, a flat panel detector, etc. is mounted at the other end, opposite to the radiation source 16, of the C-arm mount 14. The body 7 of the patient 9 is positioned on a table such that a region of interest of the body 7 (e.g., a region where the medical insert 5 is to be inserted or navigated in the body 7 of the patient 9) is positioned in-between the radiation source 16 and the image acquisition module 18 of the imaging unit 10 such that radiations 17 are allowed to pass through the region of interest. The imaging unit 10 generates the image signals by sensing the field of view 12 (e.g., by aiming radiations 17 from the radiation source 16 towards the region of interest of the body 7 of the patient 9 and by detecting, at the image acquisition module 18, the radiations 17 transmitted through the body 7 of the patient 9). The image signals generated from the imaging unit 10 may be visualized on a display unit 80.

During the navigation of the medical insert 5 inside the body 7 of the patient 9, when the medical insert 5 is present in the field of view 12 of the imaging unit 10, the medical insert 5 is visualized in the display unit 80 along with surroundings formed by the body 7 of the patient 9, as shown in FIG. 2. The field of view 12 of the imaging unit 10 is movable or adjustable (e.g., the field of view 12 may be adjusted such that a different region of interest along the body 7 of the patient 9 may be visualized on the display unit 80). For example, the field of view 12 of the imaging unit 10 depicted in FIG. 2 may be adjusted or moved to aim at a different region of interest in the body 7 of the patient 9 by moving the C-Arm mount 14 (e.g., the radiation source 16 and the image acquisition module 18 together along a translational motion in a plane parallel to and/or vertical to the body 7 of the patient 9). The field of view 12 of the imaging unit 10 depicted in FIG. 2 may also be adjusted or moved to aim at a different region of interest in the body 7 of the patient 9 by rotating the C-Arm mount 14 in yaw and/or pitch and/or roll motion as depicted by the arrows represented by reference numerals 21, 22 and 23, respectively.

The image processing unit 30 is communicatively connected with the imaging unit 10 (e.g., the image processing unit 30 is capable of receiving the image signals from the imaging unit 10). The communicative connection may be wired or wireless in nature. The image processing unit 30 is a processor such as a microprocessor, field programmable gate arrays, etc. and is configured to receive the image signals from the imaging unit 10. The image processing unit 30 includes a position determining module 40 and a control module 50. The position determining module 40 is configured to determine a position of the medical insert 5 relative to the desired area 13 (shown in FIG. 3) within the field of view 12. The position determining module 40 determines the position of the medical insert 5 relative to the desired area 13 by using the image signals received from the imaging unit 10. The position determining module 40 is configured to determine the position of the medical insert 5 by using edge detection techniques and/or object recognition techniques in the image signals generated by the imaging unit 10.

The edge detection techniques and/or object recognition techniques help to determine the presence of the medical insert 5 in the image signals generated by the imaging unit 10. In an exemplary embodiment, the position determining module 40 may be configured to implement an algorithm in which short line segments are identified in the generated image signals using steerable filters, an orientation-selective convolution kernel used for image enhancement and feature extraction, and/or Probabilistic Boosting Tree technique. Different types of features including Haar-like features are detected to identify segments such as edges and ridges in the image signals generated by the imaging unit 10. Subsequently, probabilities for multiple pairs of two segments are computed, and segments with highest probability are extended to form a curve. By repeating several times, the extension step to form a curve, multiple curves are obtained. The several curves obtained are modeled using Principle Component Analysis modeling. The medical insert 5 specific features are identified. This leads to recognition of the medical insert 5 in the image signals generated by the imaging unit 10. In other embodiments, other edge detection techniques and/or object recognition techniques may be used. Such edge detection techniques and/or object recognition techniques are well known in the art of image processing and thus not explained in detail herein for sake of brevity.

In an embodiment of the present technique, the position determining module 40 is configured to determine the position of the medical insert 5 by using a coordinate system 11, as depicted in FIG. 3, corresponding to the field of view 12 of the imaging unit 10. In an exemplary embodiment, the coordinate system 11 may be a Cartesian coordinate system and used to determine or mark the position of the medical insert 5 using the Cartesian coordinates. In another exemplary embodiment, the Cartesian coordinate system and the edge detection technique are simultaneously used to determine the position of the medical insert 5 relative to the desired area 13 in the field of view 12 of the imaging unit 10.

The control module 50 is configured to generate a control signal based on the position of the medical insert 5 relative to the desired area 13 within the field of view 12 as determined by the position determining module 40. The functioning of the position determining module 40 and the control module 50 are explained in further detail later in reference to FIGS. 3 to 8.

The drive unit 60 is in communication with the image processing unit 30. The drive unit 60 is configured to receive the control signal from the image processing unit 30 (e.g., the control module 50 of the image processing unit 30) and to move the field of view 12 in accordance with the control signal such that the medical insert 5 is maintained within the desired area 13. The drive unit 60 may include a pivot assembly (not shown) capable of effecting motion on at least a part of the imaging unit 10 (e.g., the C-arm mount 14 of the imaging unit 10) in a translational manner along a plane parallel to and/or vertical to the body 7 of the patient 9, or in a rotational manner inducing rotation of the C-Arm mount 14 in yaw and/or pitch and/or roll motion as depicted by the arrows represented by reference numerals 21, 22 and 23, respectively. The drive unit 60 may also include a motor (not shown) such as a stepper motor to move the pivot assembly in one or more of the abovementioned motions, and/or a controller (not shown) to control the motor. In another exemplary embodiment, the drive unit 60 may be a robotic arm (not shown) capable of moving the imaging unit 10 in one or more degrees of freedom.

In an embodiment of the medical device 100, the drive unit 60 is configured to move the field of view 12 in accordance with the control signal such that the medical insert 5 is maintained at a fixed relative position within the desired area 13. In another embodiment of the medical device 100, the drive unit 60 is configured to move the field of view 12 in accordance with the control signal such that the medical insert 5 is maintained at a central location of the desired area 13.

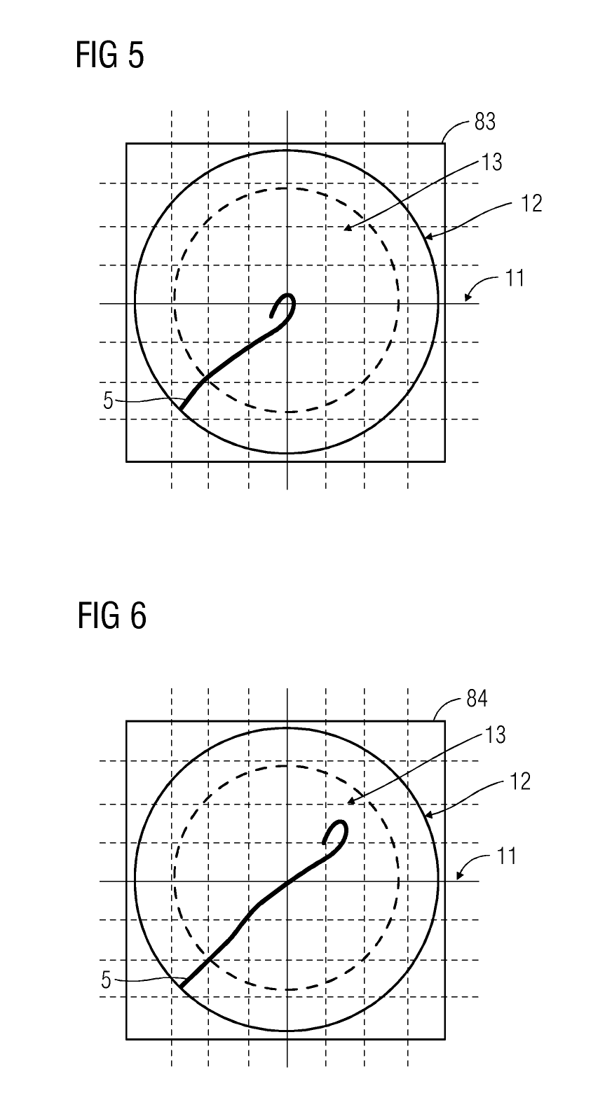

The phrase, ‘the medical insert 5 is maintained within the desired area 13’ may be that the imaging unit 10 is adjusted or moved by the drive unit 60 such that in the image signals or images successively generated by the imaging unit 10, the medical insert 5 is caused or enabled to continue within the desired area 13 in the field of view 12 of the imaging unit 10. Additionally, the phrase, “the medical insert 5 is maintained within the desired area 13” provides that if in one of the image signals or images the medical insert 5 is seen to be out of the desired area 13, then, subsequently, the drive unit 60 moves or adjusts the imaging unit 10 such that in the image signals or images generated subsequently (e.g., after the drive unit 60 has moved or adjusted the imaging unit 10), the medical insert 5 is brought back within the desired area 13 in the field of view 12 of the imaging unit 10. This is explained further with reference to some exemplary scenarios depicted by FIGS. 5, 7 and 8 that represent different positions of the medical insert 5 on the Cartesian coordinate system 11 in different images 83, 85 and 86, respectively.

In a first exemplary scenario as depicted in FIG. 5 (e.g., in the image signal or the image 83 generated by the imaging unit 10), the medical insert 5 is determined to be positioned around the center of the Cartesian coordinate system 11 within the desired area 13 in the field of view 12 of the imaging unit 10. In this scenario, the control module 50 will generate the control signal, which will not cause the drive unit 60 to move or adjust the field of view 12 (e.g., the imaging unit 10 will not be moved by the drive unit 60 and thus the field of view 12, and hence the desired area 13, will continue to have the medical insert 5 in the desired area 13).

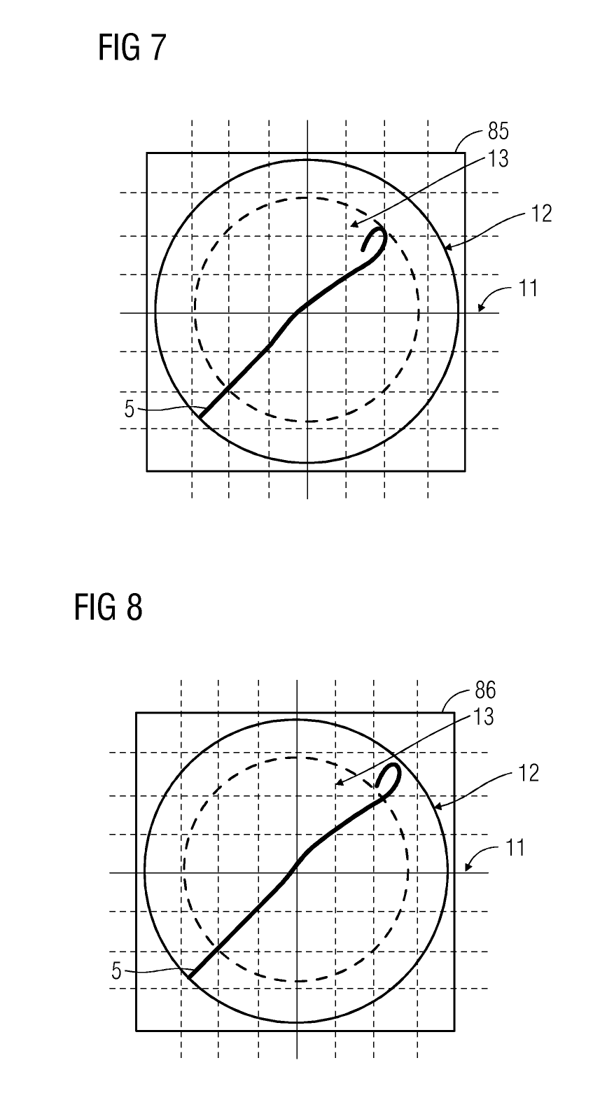

In a second exemplary scenario as depicted in FIG. 7 (e.g., in the image signal or the image 85 generated by the imaging unit 10), the medical insert 5 is determined to be positioned around an edge of the desired area 13 in the field of view 12 of the imaging unit 10. In this scenario, further subsequent navigation of the medical insert 5 may cause a problem by leading the medical insert 5 outside the field of view 12 of the imaging unit 10. To avoid occurrence of the problem, the control module 50 will generate the control signal, which will cause the drive unit 60 to move or adjust the field of view 12 such that the medical insert 5 is moved inwards inside the desired area 13 towards the center of the Cartesian coordinate system 11. This is achieved by moving the imaging unit 10 by the drive unit 60 to adjust the field of view 12, and hence the desired area 13.

In a third exemplary scenario as depicted in FIG. 8 (e.g., in the image signal or the image 86 generated by the imaging unit 10), the medical insert 5 is determined to be positioned beyond the edge of the desired area 13 and outside the desired area 13 in the field of view 12 of the imaging unit 10. In this scenario, the probability is very high that further subsequent navigation of the medical insert 5 may cause a problem by leading the medical insert 5 completely outside the field of view 12 of the imaging unit 10. To avoid occurrence of the problem, the control module 50 will generate the control signal, which will cause the drive unit 60 to move or adjust the field of view 12 such that the medical insert 5 is moved inwards inside the desired area 13 towards the center of the Cartesian coordinate system 11. This is achieved by moving the imaging unit 10 by the drive unit 60 to adjust the field of view 12, and hence the desired area 13.

Thus, by the medical device 100, the imaging unit 10 is adjusted or moved in an automated way by the action of the drive unit 60 controlled by the control module 50 via the control signal generated by the control module 60. This depends on the position of the medical insert 5 as determined by the position determining module 40 of the image processing unit 30 using the image signals generated by the imaging unit 10.

The functioning of the image processing module 30 is further explained hereinafter with the help of the embodiments shown in FIGS. 3 to 8. As mentioned earlier, FIG. 3 schematically represents the image 81 depicting an instance before the medical insert 5 is visualized in the field of view 12 of the imaging unit 10 (e.g., when the medical insert 5 is not present in the region of interest in the body 7 of the patient 9). FIGS. 4, 5, 6, 7 and 8 schematically represent exemplary embodiments of images 82, 83, 84, 85, and 86 depicting the field of view 12 and the medical insert 5 within the body 7 of the patient 9 at different positions relative to the desired area 13.

In accordance with aspects of the present technique, in an exemplary embodiment of the medical device 100, the image signals include a series of images 82, 83, 84, 85, 86 of the medical insert 5 inside the body 7 of the patient 9 at different positions within the field of view 12. This is exemplified in FIGS. 4 to 8, for the purpose of explanation. In another exemplary embodiment, the series of images 82, 83, 84, 85, 86 may be a time series (e.g., a series formed by the image signals generated by the imaging unit 10 at regular time intervals during the navigational procedure of the medical insert 5 inside the body 7 of the patient 9).

In an exemplary embodiment of the medical device 100, the position determining module 40 is configured to determine a change in position of the medical insert 5 relative to the desired area 13 within the field of view 12 by using at least a first image and a second image of the series. In this embodiment of the medical device 100, the control module 50 is configured to generate the control signal based on the change in position of the medical insert 5 relative to the desired area 13 within the field of view 12. In a related embodiment, the position determining module 40 is configured to detect a first position of the medical insert 5 within the field of view 12 from the first image of the series. The position determining module 40 is also configured to detect a second position of the medical insert 5 within the field of view 12 from the second image of the series, and to compare the first position and the second position to determine the change in position of the medical insert 5 relative to the desired area 13 within the field of view 12. In another exemplary embodiment, the position determining module 40 is configured to use a technique of subtraction between the first image and the second image to compare the first position and the second position of the medical insert 5. The technique of subtraction (e.g., an image subtraction technique or pixel subtraction technique), a process where the digital numeric value of one pixel or whole image is subtracted from another image for obtaining changes between two images, is a well known and prevalently used technique of image processing and hence is not explained in further detail herein for sake of brevity.

The determination, by the position determining module 40, of the change in position of the medical insert 5 relative to the desired area 13 and the generation of the control signal, by the control module 50, based on the change in position of the medical insert 5 relative to the desired area 13 has been further explained hereinafter with reference to some exemplary cases depicted by combinations of FIGS. 4, 5, 6, 7 and 8 that represent different positions of the medical insert 5 on the Cartesian coordinate system 11 in different images 82, 83, 84 85 and 86, respectively.

By a combination of FIG. 4 and FIG. 5, a first exemplary case is presented. In this exemplary case, the first image is the image 82 depicted in FIG. 4, and the second image is image 83 depicted in FIG. 5. For the purpose of this example, it is assumed that during the navigation of the medical insert 5 inside the body 7 of the patient 9, the scenario of FIG. 5 is subsequent to the scenario of FIG. 4. In the first image 82 generated by the imaging unit 10, the medical insert 5 is detected to be in the first position (e.g., around the edge of the desired area 13 in the field of view 12 of the imaging unit 10). In the second image 83 generated by the imaging unit 10, the medical insert 5 is detected to be in the second position (e.g., around the center of the desired area 13 in the field of view 12 of the imaging unit 10). The first and the second positions of the medical insert 5 may be detected by using edge detection technique or object recognition technique as mentioned earlier. The first and the second positions of the medical insert 5 may be detected on a Cartesian coordinate system 11. Thus, by comparing the first and the second images 82, 83 (e.g., by subtracting the first image 82 from the second image 83), the change in position of the medical insert 5 is determined by the position determining module 40. The change in position of the medical insert 5 also provides an indication of a direction of movement of the medical insert 5 relative to the desired area 13. Since, in the present exemplary case, the changed position of the medical insert 5 (e.g., the position in the second image 83) is around the center of the desired area 13 and since the direction of movement of the medical insert 5 is from the edge of the desired area 13 towards the center of the desired area 13, in this exemplary case, the control module 50 will generate the control signal that will not cause the drive unit 60 to move or adjust the field of view 12 (e.g., the imaging unit 10 will not be moved by the drive unit 60). Thus, the field of view 12, and hence the desired area 13, will continue to have the medical insert 5 in the desired area 13.

By a combination of FIG. 6 and FIG. 7, a second exemplary case is presented. In this exemplary case (e.g., the first image is the image 84 depicted in FIG. 6, and the second image is the image 85 depicted in FIG. 7). For the purpose of this example, it is assumed that during the navigation of the medical insert 5 inside the body 7 of the patient 9, the scenario of FIG. 7 is subsequent to the scenario of FIG. 6. In the first image 84 generated by the imaging unit 10, the medical insert 5 is detected to be in the first position (e.g., midway between the center and the edge of the desired area 13). In the second image 85 generated by the imaging unit 10, the medical insert 5 is detected to be in the second position (e.g., around the edge of the desired area 13 in the field of view 12 of the imaging unit 10). Thus, by comparing the first and the second images 84, 85 (e.g., by subtracting the first image 84 from the second image 85), the change in position of the medical insert 5 is determined by the position determining module 40. The change in position of the medical insert 5 also provides an indication of a direction of movement of the medical insert 5 relative to the desired area 13. Since, in the present exemplary case, the changed position of the medical insert 5 (e.g., position in the second image 85) is around the edge of the desired area 13 and since the direction of movement of the medical insert 5 is from the center of the desired area 13 towards the edge of the desired area 13, in this exemplary case, further subsequent navigation of the medical insert 5 may cause problem by leading the medical insert 5 outside the field of view 12 of the imaging unit 10. To avoid occurrence of the problem, the control module 50 will generate the control signal, which will cause the drive unit 60 to move or adjust the field of view 12 such that the medical insert 5 is moved inwards inside the desired area 13 towards the center of the Cartesian coordinate system 11. This is achieved by moving the imaging unit 10 by the drive unit 60 to adjust the field of view 12, and hence the desired area 13.

By a combination of FIG. 6 and FIG. 5, a third exemplary case is presented. In this exemplary case, the first image is the image 84 depicted in FIG. 6, and the second image is the image 83 depicted in FIG. 5. For the purpose of this example, it is assumed that during the navigation of the medical insert 5 inside the body 7 of the patient 9, the scenario of FIG. 5 is subsequent to the scenario of FIG. 6. In the first image 84 generated by the imaging unit 10, the medical insert 5 is detected to be in the first position (e.g., midway between the center and the edge of the desired area 13). In the second image 83 generated by the imaging unit 10, the medical insert 5 is detected to be in the second position (e.g., around the center of the desired area 13 in the field of view 12 of the imaging unit 10). Thus, by comparing the first and the second images 84, 83 (e.g., by subtracting the first image 84 from the second image 83), the change in position of the medical insert 5 is determined by the position determining module 40. The change in position of the medical insert 5 also provides an indication of a direction of movement of the medical insert 5 relative to the desired area 13. Since, in the present exemplary case, the changed position of the medical insert 5 (e.g., the position in the second image 83) is around the center of the desired area 13 and since the direction of movement of the medical insert 5 is from the edge of the desired area 13 towards the center of the desired area 13, in this exemplary case, further subsequent navigation of the medical insert 5 will not cause problems because the medical insert 5 is well within the desired area 13. Thus, the control module 50 will generate the control signal, which will not cause the drive unit 60 to move or adjust the field of view 12 (e.g., the imaging unit 10 will not be moved by the drive unit 60). Thus, the field of view 12, and hence the desired area 13, will continue to have the medical insert 5 in the desired area 13.

By a combination of FIG. 5 and FIG. 8, a fourth exemplary case is provided. In this embodiment, the first image is the image 83 depicted in FIG. 5, and the second image is the image 86 depicted in FIG. 8. For the purpose of this example, it is assumed that during the navigation of the medical insert 5 inside the body 7 of the patient 9, the scenario of FIG. 8 is subsequent to the scenario of FIG. 5. Since, in the present exemplary case, the changed position of the medical insert 5 (e.g., the position in the second image 85) is outside of the desired area 13 and since the direction of movement of the medical insert 5 is from the center of the desired area 13 towards the outside of the desired area 13, in this exemplary case, further subsequent navigation of the medical insert 5 may cause a serious problem by leading the medical insert 5 completely outside the field of view 12 of the imaging unit 10. To avoid occurrence of the problem, the control module 50 will generate the control signal, which will cause the drive unit 60 to move or adjust the field of view 12 such that the medical insert 5 is moved inwards inside the desired area 13 towards the center of the Cartesian coordinate system 11. This is achieved by moving the imaging unit 10 by the drive unit 60 to adjust the field of view 12, and hence the desired area 13.

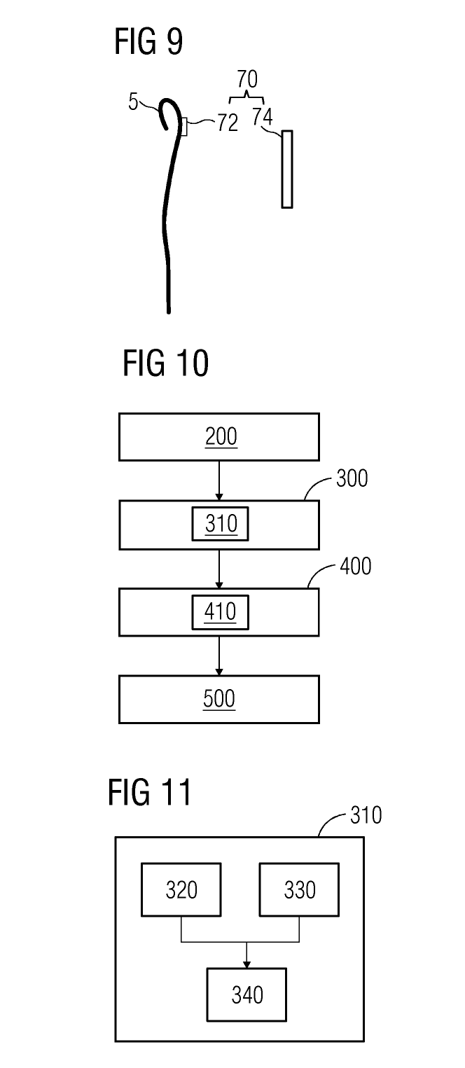

FIG. 9, in combination with FIG. 2, schematically represents yet another exemplary embodiment of the medical device 100, which includes a motion sensing unit 70. The motion sensing unit 70 is configured to sense a movement of the medical insert 5 inside the body 7 of the patient 9 and to induce the imaging unit 10 to generate at least one image signal by sensing the field of view 12 in response to the movement of the medical insert 5 inside the body 7 of the patient 9. The motion sensing device 70 may be any device to detect the movement of the medical insert 5 inside the body 7 of the patient 9 (e.g., the motion sensing unit 70 may be a transmitter 72—a receiver 74 pair such as RF (radio frequency) transmitter—receiver pair). As depicted in FIG. 9, the transmitter 72 may be positioned on the medical insert 5, and the receiver 74 may be positioned outside the patient's body 7 (e.g., at a fixed spot (not shown) in the exam room). Thus, a subsequent image signal is generated by the imaging unit 10 only when the medical insert 5 is moved from a given position.

FIG. 10 is a flow diagram depicting an exemplary embodiment of a method 1000 for assisting navigation of the medical insert 5 inside the body 7 of the patient 9, and FIG. 11 is a flow diagram depicting another exemplary embodiment of the method 1000. In accordance with aspects of the present technique. FIGS. 10 and 11 are described in combination with FIGS. 1 to 9.

In the method 1000, image signals are generated by sensing the field of view 12 of the imaging unit 10 in act 200. In act 300, a position of the medical insert 5 is determined relative to the desired area 13 within the field of view 12. The position of the medical insert 5 relative to the desired area 13 is determined by using the image signals. The position of the medical insert 5 is determined by a processing element such as a microprocessor (e.g., the image processing unit 30). Subsequently, in act 400, the control signal is generated based on the position of the medical insert 5 relative to the desired area 13 within the field of view 12. The control signal is generated by a processing element such as a microprocessor (e.g., the image processing unit 30). In act 500, the field of view 12 of the imaging unit 10 is automatically moved in accordance with the control signal such that the medical insert 5 is maintained within the desired area 13. The field of view 12 may be automatically moved by using the drive unit 60. The imaging unit 10, the image processing unit 30 and the drive unit 60 are similar to those described in reference to FIGS. 1 to 9.

In the method 1000, the position of the medical insert 5 may be determined by using an edge detection technique and/or an object recognition technique, as described earlier. A coordinate system 11 (e.g., a Cartesian coordinate system) may be used in determining the position of the medical insert 12 relative to the desired area 13.

In an exemplary embodiment of the method 1000, in act 500, the field of view 12 of the imaging unit 10 is automatically moved in accordance with the control signal such that the medical insert 5 is maintained at a fixed relative position within the desired area 13. In another exemplary embodiment of the method 1000, in act 500, the field of view 12 of the imaging unit 10 is automatically moved in accordance with the control signal such that the medical insert 5 is maintained at a central location of the desired area 13.

In an exemplary embodiment of the method 1000, in act 200 and act 300, the image signals include a series of images 82, 83, 84, 85, 86 of the medical insert 5 inside the body 7 of the patient 9 at different positions within the field of view 12. In another exemplary embodiment of the method 1000, the act 300 includes act 310 of determining a change in position of the medical insert 5 relative to the desired area 13 within the field of view 12 by using at least a first image and a second image of the series. In this embodiment of the method 1000, the act 400 includes act 410 of generating the control signal based on the change in position of the medical insert 5 relative to the desired area 13 within the field of view 12. In a related exemplary embodiment of the method 1000, the act 310 includes act 320 of detecting a first position of the medical insert 5 within the field of view 12 from the first image of the series, act 330 of detecting a second position of the medical insert 5 within the field of view 12 from the second image of the series, and act 340 of comparing the first position and the second position to determine the change in position of the medical insert 5 relative to the desired area 13 within the field of view 12. In the method 1000, the first position and the second position may be compared in act 340 by performing subtraction between the first image and the second image.

In an exemplary embodiment of the method 1000, before the act 200, a movement of the medical insert 5 inside the body 7 of the patient 9 is sensed, which in turn induces the imaging unit 10 to generate at least one image signal by sensing the field of view 12 in response to the movement of the medical insert 5 inside the body 7 of the patient 9.

It is to be understood that the elements and features recited in the appended claims may be combined in different ways to produce new claims that likewise fall within the scope of the present invention. Thus, whereas the dependent claims appended below depend from only a single independent or dependent claim, it is to be understood that these dependent claims can, alternatively, be made to depend in the alternative from any preceding or following claim, whether independent or dependent, and that such new combinations are to be understood as forming a part of the present specification.

Although the present technique has been described with reference to specific embodiments, this description is not meant to be construed in a limiting sense. Various modifications of the disclosed embodiments, as well as alternate embodiments of the technique, will become apparent to persons skilled in the art upon reference to the description of the technique. It is therefore contemplated that such modifications can be made without departing from the embodiments of the present technique as defined.

1. A medical device for assisting navigation of a medical insert inside a body of a patient, the medical device comprising:
an imaging unit having a field of view and configured to generate image signals by sensing the field of view;
an image processing unit in communication with the imaging unit and configured to receive the image signals from the imaging unit, wherein the image processing unit comprises:
a position determining module configured to determine a position of the medical insert relative to a desired area within the field of view, wherein the position of the medical insert relative to the desired area is determined by using the image signals; and
a control module configured to generate a control signal based on the position of the medical insert relative to the desired area within the field of view; and
a drive unit in communication with the image processing unit, wherein the drive unit is configured to receive the control signal from the image processing unit and to move the field of view in accordance with the control signal such that the medical insert is maintained within the desired area.

2. The medical device of claim 1, wherein the image signals comprise a series of images of the medical insert inside the body of the patient at different positions within the field of view.

3. The medical device of claim 2, wherein the position determining module is configured to determine a change in position of the medical insert relative to the desired area within the field of view by using at least a first image and a second image of the series of images, and
wherein the control module is configured to generate the control signal based on the change in position of the medical insert relative to the desired area within the field of view.

4. The medical device of claim 3, wherein the position determining module is configured to:
detect a first position of the medical insert within the field of view from the first image of the series;
detect a second position of the medical insert within the field of view from the second image of the series; and
compare the first position and the second position to determine the change in position of the medical insert relative to the desired area within the field of view.

5. The medical device of claim 4, wherein the position determining module is configured to subtract between the first image and the second image to compare the first position and the second position of the medical insert.

6. The medical device of claim 1, wherein the position determining module is configured to determine the position of the medical insert by using an edge detection technique.

7. The medical device of claim 1, wherein the position determining module is configured to determine the position of the medical insert by using a coordinate system corresponding to the field of view of the imaging unit.

8. The medical device of claim 1 further comprising a motion sensing unit configured to:
sense a movement of the medical insert inside the body of the patient; and
induce the imaging unit to generate at least one image signal by sensing the field of view in response to the movement of the medical insert inside the body of the patient.

9. The medical device of claim 1, wherein the drive unit is configured to move the field of view in accordance with the control signal such that the medical insert is maintained at a fixed relative position within the desired area.

10. The medical device of claim 9, wherein the fixed relative position is at a central location of the desired area.

11. A method for assisting navigation of a medical insert inside a body of a patient, the method comprising:
generating image signals, the generating comprising sensing a field of view of an imaging unit;
determining a position of the medical insert relative to a desired area within the field of view, the determining comprising using the image signals;
generating a control signal based on the position of the medical insert relative to the desired area within the field of view; and
automatically moving the field of view of the imaging unit in accordance with the control signal such that the medical insert is maintained within the desired area.

12. The method of claim 11, wherein the image signals comprise a series of images of the medical insert inside the body of the patient at different positions within the field of view.

13. The method of claim 12, wherein determining the position of the medical insert relative to the desired area within the field of view comprises determining a change in position of the medical insert relative to the desired area within the field of view by using at least a first image and a second image of the series of images, and
wherein generating the control signal comprises generating the control signal based on the change in position of the medical insert relative to the desired area within the field of view.

14. The method of claim 13, wherein determining the change in position of the medical insert comprises:
detecting a first position of the medical insert within the field of view from the first image of the series of images;
detecting a second position of the medical insert within the field of view from the second image of the series of images; and
comparing the first position and the second position to determine the change in position of the medical insert relative to the desired area within the field of view.

15. The method of claim 14, wherein comparing the first position and the second position comprises subtracting between the first image and the second image.

16. The method of claim 11, wherein determining the position of the medical insert comprises using an edge detection technique.

17. The method of claim 11, wherein determining the position of the medical insert comprises using a co-ordinate system corresponding to the field of view of the imaging unit.

18. The method of claim 11, further comprising:
sensing a movement of the medical insert inside the body of the patient; and
inducing the imaging unit to generate at least one image signal, the inducing comprising sensing the field of view in response to the movement of the medical insert inside the body of the patient.

19. The method of claim 11, further comprising automatically moving the field of view of the imaging unit in accordance with the control signal such that the medical insert is maintained at a fixed relative position within the desired area.

20. The method of claim 19, wherein the fixed relative position is at a central location of the desired area.
